(19) United States
(12) Patent Application Publication
(10) Pub. No.: US 2007/0092537 A1
(43) Pub. Date: Apr. 26, 2007

(54) Compositions and methods for the treatment of canine influenza virus disease

(75) Inventors: Yu-Wei Chiang, Fort Dodge, IA (US); Hsien-Jue Chu, Fort Dodge, IA (US); Michael A. Gill, Fort Dodge, IA (US); Kim Gugisberg, Fort Dodge, IA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/582,652

(22) Filed: Oct. 18, 2006

Related U.S. Application Data
(60) Provisional application No. 60/728,662, filed on Oct. 20, 2005.
(60) Provisional application No. 60/735,290, filed on Nov. 10, 2005.

(51) Int. Cl. A61K 39/145

(52) U.S. Cl. 424/209.1

(57) ABSTRACT

Compositions, including vaccine compositions, and methods for treating, preventing or ameliorating canine influenza virus (CIV) disease by utilizing one or more canine influenza virus (CIV) or equine influenza virus (EIV) strain or immunogens thereof are set forth herein. Also set forth are challenge models useful in assessing the efficacy of a composition against canine influenza virus, comprising an equine influenza virus (EIV) or canine influenza virus (CIV) strain or immunogens thereof.

### CROSS-REFERENCE TO RELATED APPLICATIONS

This application claims priority from copending provisional application No. 60/728,662 filed on Oct. 20, 2005, and provisional application No. 60/735,290 filed Nov. 10, 2005. The contents of the aforementioned applications are incorporated by reference herein in their entireties.

### FIELD OF THE INVENTION

The present invention relates to compositions and methods that provide protection against influenza virus disease, including canine influenza virus (CIV) disease. The invention further relates to compositions containing an equine influenza virus (EIV) strain or immunogenic portions thereof and compositions containing a canine influenza virus (CIV) strain or immunogenic portions thereof. The invention further relates to CIV strains, EIV strains and immunogenic portions of CIV and EIV strains that can be used in challenge models for the evaluation of the immunogenicity or efficacy of canine influenza vaccines in dogs or other susceptible species.

### BACKGROUND OF THE INVENTION

Canine Influenza Virus (CIV) disease, or canine flu, is a highly contagious affliction of dogs that is marked by severe flu symptoms of respiratory distress, coughing and fever. The virus was first identified in racing greyhounds and appears to have been the cause of significant respiratory disease on canine tracks throughout the United States for the last few years. The most recent cases have occurred in dog breeds other than greyhounds in shelters, boarding facilities, and veterinary clinics throughout the country. All dogs, regardless of breed or age, are susceptible to infection and do not have naturally acquired or vaccine-induced immunity. While most dogs that become infected experience a milder form of influenza, some develop a more acute disease with clinical signs of pneumonia. (See www.cdc.gov/od/oc/media/transcripts/t050926.htm and Yoon, K-J, Cooper, V L, Schwartz, K J, et al. (2005) Emerging Infectious Diseases 11: 1974-1976, which are hereby incorporated by reference). Among the latter group, the mortality rate is 1 to 5 percent.

At least one form of the virus has been sequenced at the Centers for Disease Control (CDC) as subtype H3N8 and was found to be closely related to equine influenza virus. Researchers at the CDC suspect that a change of 8 to 10 amino acids in the Hemagglutinin “H” gene may be responsible for the ability of the virus to infect dogs.

This highly transmissible virus and newly emerging respiratory pathogens in dogs cause a clinical syndrome that mimics “kennel cough.” Canine influenza virus infections are frequently mistaken for infections due to the Bordetella bronchiseptica/parainfluenza virus complex. Virtually 100 percent of exposed dogs become infected; nearly 80 percent have clinical signs. There are two general clinical syndromes—the milder syndrome and a more severe pneumonia syndrome. The milder disease syndrome occurs in most dogs. The incubation period is two to five days after exposure before clinical signs appear. Infected dogs may shed virus for seven to 10 days from the initial day of clinical signs. Nearly 20 percent of infected dogs will not display clinical signs and become the silent shedders and spreaders of the infection.

In the milder disease, the most common clinical sign is a cough that persists for 10-21 days despite therapy with antibiotics and cough suppressants. Most dogs have a soft, moist cough, while others have a dry cough similar to that induced by the Bordetella bronchieseptical/parainfluenza virus infection. Many dogs have purulent nasal discharge and a low-grade fever. The nasal discharge likely represents a secondary bacterial infection that quickly resolves following treatment with a broad-spectrum, bacterial antibiotic.

Some dogs develop a more severe disease with clinical signs of pneumonia, such as a high fever (104° F. to 106° F.) and increased respiratory rate and effort. Thoracic radiographs may show consolidation of lung lobes. Dogs with pneumonia often have a secondary bacterial infection and have responded best to a combination of broad-spectrum, bactericidal antibiotics and maintenance of hydration with intravenous fluid therapy.

At this time, there is no known vaccine for canine influenza virus. This virus is spread by aerosolized respiratory secretions, contaminated inanimate objects, and even by people moving back and forth between infected and uninfected dogs. CIV is an enveloped virus that is most likely killed by routine disinfectants such as quaternary ammoniums and 10 percent bleach. Because the virus is highly contagious and all dogs are susceptible to infection, veterinarians, boarding facilities, shelters, pet stores, and pet owners desire an effective means to combat this disease and spare their animals the suffering, and possible death, associated therewith.

What is needed in the art, therefore, are effective compositions and methods to treat, prevent, and/or ameliorate influenza virus disease, including canine influenza virus disease. Also needed are novel immunogens that may be utilized in vaccines against CIV. Further needed are novel strains that are useful in challenge models for demonstrating the efficacy of a particular vaccine against canine influenza. In addition, the art has shown a need for vaccines against canine influenza disease that are derived from canine influenza strains and/or non-canine influenza strains, such as equine influenza strains.

### SUMMARY OF THE INVENTION

The present invention achieves these and other related needs by providing compositions and methods for the treatment, prevention, and/or amelioration of disease associated with canine influenza virus infection.

Thus, within one embodiment, the present invention provides compositions for the treatment and/or protection of dogs against disease associated with canine influenza virus (CIV) wherein the compositions comprise one or more equine influenza virus (EIV) strain and/or one or more immunogenic portion of one or more EIV strain. Immunogenic portions of an EIV strain include, for example, an EIV protein, an EIV peptide, or any other portion of an EIV strain that evokes an immune response. EIV strains suitable for use in compositions, including vaccine compositions, described herein may be isolated from a canine having clinical symptoms of influenza disease.

Within other embodiments, the present invention provides compositions for the treatment and/or protection of dogs against disease associated with canine influenza virus (CIV) wherein the compositions comprise one or more canine influenza virus (CIV) strain and/or one or more immunogenic portion of one or moe CIV strain. Immunogenic portions of a CIV strain include, for example, a CIV protein, a CIV peptide, or any other portion of a CIV strain that evokes an immune response. CIV strains suitable for use in compositions, including vaccine compositions, described herein may be isolated from a canine having clinical symptoms of influenza disease.

Within other embodiments, the present invention provides methods for preparing compositions against influenza virus, including CIV, using a strain of EIV and/or immunogenic portion(s) thereof. In some embodiments, the strain of EIV is isolated from one or more canine infected with a strain of EIV. In some aspects of these embodiments, the strain of EIV is pathogenic.

Within other embodiments, the present invention provides methods for preparing compositions against influenza virus, including CIV, using a strain of CIV and/or immunogenic portion(s) thereof. In some embodiments, the strain of CIV is isolated from one or more canine infected with a strain of CIV. In some aspects of these embodiments, the strain of CIV is pathogenic.

Further embodiments of the present invention provide strains of EIV for use in compositions, including vaccine compositions, that may, for example, be used for the treatment of disease associated with influenza virus infection, including CIV infection. For example, strains of EIV may be used in compositions used for the treatment of canine influenza. Further embodiments of the present invention provide immunogenic portions of an EIV strain that may be used for the treatment of disease associated with influenza virus infection, including CIV infection.

Still further embodiments of the present invention provide strains of CIV for use in compositions, including vaccine compositions, that may, for example, be used for the treatment of disease associated with influenza virus infection, including CIV infection. For example, strains of CIV may be used in compositions used for the treatment of canine influenza. In some aspects of these embodiments, the strains of CIV are highly efficacious strains. Further embodiments of the present invention provide immunogenic portions of a CIV strain that may be used for the treatment of disease associated with influenza virus infection, including CIV infection.

In yet further embodiments, the present invention provides methods for the protection of canine species against influenza virus infection, including CIV infection, which methods comprise the step of administering a composition, such as a vaccine composition, that is derived from one or more isolated EIV strain(s) and/or one or more immunogenic portion(s) of an EIV strain.

In some embodiments, the present invention provides methods for the protection of canine species against influenza virus infection, including CIV infection, which methods comprise the step of administering a composition, such as a vaccine composition, that is derived from one or more isolated CIV strain(s) and/or one or more immunogenic portion(s) of a CIV strain.

In still further embodiments, the present invention provides challenge models for demonstrating the efficacy of compositions, including vaccine compositions, against canine influenza virus wherein the challenge model utilizes one or more isolated equine influenza virus strain. In some embodiments, the challenge models may utilize one or more immunogenic portion of one or more EIV strain. In some embodiments, the challenge models may utilize one or more canine influenza virus strain. In some embodiments, the challenge model may utilize one or more immunogenic portion of one or more CIV strain.

These and other embodiments, features, and advantages of the invention will become apparent from the detailed description and the appended claims set forth herein below. All literature and patent references cited throughout the application are hereby incorporated by reference in their entireties.

### DETAILED DESCRIPTION OF THE INVENTION

As indicated above, the present invention is based upon the observation that certain strain(s) of canine influenza virus (CIV) and equine influenza virus (EIV) may be suitably employed in compositions, including vaccine compositions, for the treatment, prevention, and/or amelioration of disease associated with infection by one or more strain(s) of influenza virus, including canine influenza virus (CIV). As used herein, the term “canine” refers to any species of wild or domesticated dog known in the art while the term “equine” refers to any species of wild or domesticated horse known in the art.

A suitable immunogen for use in compositions and vaccine compositions suitable for the treatment of influenza virus disease, including CIV disease, may be isolated from one or more canine infected with one or more influenza virus, such as CIV or EIV strains. Typically, the immunogen comprises one or more CIV or EIV strain that may be isolated from tissue, blood, discharge, or saliva samples of CIV or EIV infected dogs by techniques known in the art. The selected CIV or EIV strain may be used to infect canine cells in culture such as, for example, cultured canine kidney cells, from which a master seed virus is propagated and harvested. For example, a CIV or EIV strain may be used to infect a canine cell line such as Madin Darby Canine Kidney (MDCK; ATCC #CCL 34, NBL-2) cells. Alternatively, CIV or EIV strains may be cultured in other cells or suitable media available in the art such as, for example, chicken embryonated eggs. Suitable immunogens for use in compositions and vaccine compositions suitable for the treatment of influenza virus disease, including CIV disease, also include one or more immunogenic portions of one or more EIV or CIV strain.

The CIV or EIV strain may be isolated from those infected animals that exhibit clinical symptoms of flu disease such as cough, fever, respiratory distress, discharge, and/or other associated symptom(s). An exemplary immunogen described herein may be isolated from the Ohio 03 strain of EIV that has demonstrated the capacity to infect and cause flu symptoms in dogs. Another exemplary immunogen described herein may be isolated from the H3N8 strain of CIV, which has also demonstrated the capacity to infect and cause flu symptoms in dogs. Another exemplary immunogen may be isolated from the Kentucky 97 strain of EIV.

In certain embodiments, the CIV or EIV strain used in the context of the present invention will be a strain that causes virus shedding and/or clinical symptoms (e.g., sneezing, coughing, fever, respiratory distress, nasal discharge, etc.) in greater than about 50% of animals challenged with the virus. For example, the CIV or EIV strain may cause virus shedding and/or clinical symptoms in greater than about 55%, 60%, 65%, 70%, 75%, 80%, 85%, 90% or 95% of animals challenged with the virus. In certain embodiments, the CIV or EIV strain used in the context of the present invention will be a strain that causes virus shedding and/or clinical symptoms in about 100% of animals challenged with the virus.

Immunogens that may be employed in the generation of the compositions, including vaccine compositions, described herein may be live, attenuated, or killed (inactivated) virions, such as EIV or CIV. If attenuated, then serial passaging of the virus using available technology may be recommended to lessen its virulence, while retaining its immunogenicity. Whole or subunit influenza virions may be inactivated by conventional means such as, for example, through chemical inactivation using one or more chemical inactivating agents including, but not limited to, one or more of binary ethyleneimine, beta-propiolactone, formalin, gluteraldehyde, and/or sodium dodecyl sulfate. Virions may also be inactivated by heat or psoralen in the presence of ultraviolet light. Immunogens may also be derived from highly pathogenic EIV strains that elicit clinical influenza symptoms in dogs. Other suitable CIV or EIV immunogens include viral proteins or peptides that are capable of eliciting an effective immune response against CIV disease when administered as part of a composition as described herein.

Also contemplated for use herein are nucleic acids isolated from CIV or EIV in fluids or tissues of canine species exhibiting influenza symptoms following infection with CIV or EIV. Such fluids or tissues include, but are not limited to, cerebral spinal fluid or sections of spinal cord or brain. Nucleic acids, typically DNA, encoding a CIV or an EIV protein immunogen may be cloned into a suitable plasmid vector and transformed into one or more suitable cell(s), such as E. coli, to obtain a master seed. The master seed may then be cultured, passaged, and harvested and the plasmid isolated using techniques available to the skilled artisan.

Compositions, including vaccine compositions, which are effective in eliciting an immune response against CIV disease utilize one or more of the immunogen(s) herein provided. The effective immunizing amount of the CIV or EIV immunogen may vary and may be any amount sufficient to evoke an immune response and, within certain aspects, provide immunological protection against subsequent challenge with one or more strain of canine influenza virus. In those aspects of the compositions of the present invention wherein the immunogen is one or more CIV or EIV virion or portion thereof, dosage units comprise at least about 1×10⁴ TCID₅₀ of killed, attenuated, or inactivated virion or immunogen derived therefrom or a mixture thereof. Typically, dosage units comprise at least about 1×10⁶ TCID₅₀, more typically at least about 1×10⁷ TCID₅₀ or at least about 5×10⁷ TCID₅₀ of killed or inactivated whole or subunit CIV or EIV virion or portion thereof. In certain aspects of these embodiments dosage units may comprise as much as 1×10⁹ TCID₅₀ or more of killed or inactivated whole or subunit CIV or EIV virion or portion thereof. Thus, a suitable range of killed or inactivated whole or subunit CIV or EIV virion or portion thereof is between about 1×10⁴ TCID₅₀ and about 1×10⁹ TCID₅₀.

In those aspects of the present invention wherein the immunogen is encoded by one or more CIV or EIV nucleic acid, as indicated above, it is contemplated that about 50 to 3,000 micrograms (μg) of plasmid DNA may be utilized in one dosage unit of the vaccine composition. More typically, about 100 to about 1,000 μg or about 100 to 250 μg of plasmid DNA may be used.

The composition may contain a pharmacologically acceptable carrier available in the art. The composition may be in aqueous or non-aqueous form, or may be in the form of an emulsion, for example, a water-in-oil emulsion.

Compositions, including vaccine compositions, of the present invention may be adjuvanted using one or more adjuvant(s) available in the art. As used herein the term “adjuvant” refers to any component that improves the body's response to a vaccine. The adjuvant typically comprises about 0.1% to about 50% vol/vol of the vaccine compositions of the invention, more typically about 1% to about 50% of the vaccine, and even more typically about 1% to about 20% thereof. Amounts of about 4% to about 10% may be even more typical. Suitable adjuvants include, but are not limited to, aluminum hydroxide, which is often used in aqueous-based formulations, as well as oil-based formulations such as SP oil, mineral oil, squalane, squalene, and other oils. Another suitable adjuvant is an EMA (ethylene maleic acid)/Neocryl formulation. Other metabolizable oils that may be employed for use in the compositions of the invention include Emulsigen (MPV Laboratories, Ralston, NZ), Montanide 264,266,26 (Seppic SA, Paris, France), and also peanut oil and other vegetable-based oils, or other metabolizable oils that can be shown to be suitable as an adjuvant in veterinary vaccine practice.

In addition, compositions may additionally or alternatively contain one or more other diluents, excipients, and/or preservatives to assist in the formulation thereof. For example, surfactants and wetting agents may be utilized in the compositions in amounts of about 0.1% to about 25%, more typically about 1% to about 10%, and even more typically about 1% to about 3% by volume of the adjuvant. Wetting or dispersing agents may be non-ionic surfactants including, for example, polyoxyethylene/polyoxypropylene block copolymers, such as those marketed under the trademark PLURONIC® and available from BASF Corporation (Mt. Olive, N.J.). Other useful nonionic surfactants include polyoxyethylene esters such as polyoxyethylene sorbitan monooleate, which is available under the trademark TWEEN 80®. Other surfactants available in the art may also be utilized depending upon the precise nature of the composition contemplated.

Compositions, including vaccine compositions, of the invention may be administered to healthy canines in one or more dosages. At least one dosage unit per animal is contemplated herein as a vaccination regimen. In some embodiments, two or more dosage units may be especially useful. A dosage unit may typically be about 0.1 ml to about 10 ml of composition, more typically about 0.5 ml to about 5 ml, and even more typically about 1 ml to about 2 ml, with each dosage unit containing the titre of virions or quantity of immunogenic virion components described above. The skilled artisan will appreciate that a particular quantity of composition per dosage unit, as well as the total number of dosage units per vaccination regimen, may be varied and optimized, so long as an effective immunizing titre of virions or immunogenic component(s) thereof is administered to the animal. If more than one dosage is utilized, then administration of the composition is typically spaced by a period of between about two weeks and about two months.

Compositions, including vaccine compositions, may be administered parenterally, or by other suitable means. For example, compositions may be administered subcutaneously, intraperitoneally, intradermally, or via food or drinking water, or via the nasal or other soft tissue passages.

Compositions may also be combined with one or more additional immunogen(s) such as, for example, one or more immunogen(s) against other canine afflictions.

In contrast to other equine influenza virus-based vaccines available in the art, which are incapable of eliciting a substantial antibody response when administered to canines, the compositions, including vaccine compositions, herein described are capable of eliciting an immune response against canine influenza virus disease when administered to dogs. Without being bound by any mechanistic theory, it is believed that administration of a highly virulent or highly pathogenic EIV strain (such as, for example, Ohio 03) provides a suitable immunogen for use in an efficacious vaccine for influenza virus infection, including CIV, as described herein.

The following non-limiting examples are provided to illustrate various aspects of the present invention.

### EXAMPLE 1

### Challenge Model for Canine Flu Using Equine Influenza Virus

This example demonstrates the development of an experimental challenge model that mimics the emerging canine flu.

Animals

Ten (10) healthy Beagle/Mongrel dogs, 4 months old of age.

Experimental Design

Dogs were randomized, block by litter, into groups as shown in the table below using the random number generator in Microsoft® Excel.

| Group | Targeted Challenge Dose | Number of Dogs |
|---|---|---|
| 1 | 10⁷ EID₅₀ | 5 |
| 2 | 10⁶ EID₅₀ | 5 |

“EID” as used herein means egg infectious dose.

Experimental Challenge

The Ohio 03 strain of EIV was obtained from Dr. Tom Chambers of the University of Kentucky, Gluck Equine Research Center. The virus was subcultured in eggs at Fort Dodge Animal Health (FDAH) to establish an adequate volume of challenge material. On the day of challenge, the challenge virus was thawed quickly. Aliquots of the challenge virus were kept on ice throughout the challenge procedure. Each dog was challenged intranasally with an aliquot of virus (2 mL) using a nebulizer. To facilitate the challenge, the dogs were sedated according to standard methods.

Observation and Sample Collection

To establish baselines, rectal temperatures were monitored and dogs were observed for nasal discharge, ocular discharge, coughing, and dyspnea twice daily for three days prior to challenge. Thereafter, rectal temperatures were recorded and dogs were observed for the aforementioned respiratory signs twice daily for 4 days post challenge and once daily on the fifth day post challenge.

Dogs were bled on the day of challenge and on the fifth day post challenge. Nasal and pharyngeal swabs were collected daily from each dog starting 1 day prior to challenge until 5 days post challenge (DPC). At the end of the study all animals were euthanized and necropsied. Trachea, lung, thymus, tonsil, retropharyngeal lymph node, and bronchial lymph node were examined for gross pathology. In addition, samples were collected from these tissues for histopathology.

Virus Isolation

Virus shedding was determined by performing virus isolation from nasal and pharyngeal swabs using 9-11 day old embryonated eggs. Virus isolation from tissue samples was also attempted.

Titration of Challenge Virus

The actual titers of virus received by dogs in each group are shown in the table below:

Actual Challenge Dose

| | Intended Challenge | Pre- | Post- |
|---|---|---|---|
| Group | Dose | Challenge | Challenge |
| 1 | 10⁷ EID₅₀ | 10^{8.7} EID₅₀ | 10^{8.8} EID₅₀ |
| 2 | 10⁶ EID₅₀ | 10^{6.5} EID₅₀ | 10^{6.1} EID₅₀ |

Clinical Observation

Prior to the experimental challenge, all dogs were healthy and no respiratory signs were observed (see Tables 1-4). After challenge, in contrast to the findings in dogs challenged with canine flu isolate (Crawford, P. C. et al. 2005 Science 310:482-485), respiratory signs (e.g., coughing, serous nasal discharge, or sneezing) were induced in all dogs challenged with 6 logs of EIV Ohio 03 (Table 1). Two dogs in this group also developed a low-grade fever (Table 2; temperature above 103° F. and 1° F. above baseline).

Dogs challenged with 2 logs higher dose of EIV Ohio 03 exhibited fewer respiratory signs (Table 3) although there were more dogs in this group that had low-grade fever (Table 4). One speculation to explain this difference in respiratory signs is that the whopping challenge dose received by those dogs induced a quick onset of production of anti-viral interferon. This may also explain the differences in isolation of virus from tissues and shedding between these two groups as shown in Table 5. While 60% of dogs from Group 2 (6 logs) were positive for positive isolation from lung, only one dog (20%) from Group 1 (8 logs) was positive. Similar findings were observed for trachea and tonsil. A prominent difference in shedding was observed based on pharyngeal swab samples but not based on nasal swab samples. Testing of nasal swab samples collected in other time points is ongoing.

The pathogenicity of EIV Ohio 03 in dogs is well demonstrated in this study. Therefore, it is reasonable to utilize this challenge model in evaluating the efficacy of candidate canine influenza vaccine. Based on the data published by Crawford et al (Crawford, P. C. et al. 2005 Science 310:482-485), EIV Ohio 03 seems more virulent than the dog flu isolate originally isolated from the outbreak in Florida. Our data further support the theory that canine flu is due to the interspecies transfer of an equine influenza virus.

TABLE 1
RESPIRATORY SIGNS DETECTED IN DOGS CHALLENGED WITH 6 LOGS OF
EIV OHIO 03

| DPC | | | | | |
|---|---|---|---|---|---|
| (Day Post | Dogs | | | | |
| Challenge) | C5 1003 | C5 1005 | C5 1103 | C5 1106 | C5 1107 |
| Day −2 am | Normal | Normal | Normal | Normal | Normal |
| Day −2 pm | Normal | Normal | Normal | Normal | Normal |
| Day −1 am | Normal | Normal | Normal | Normal | Normal |
| Day −1 pm | Normal | Normal | Normal | Normal | Normal |
| Day 0 am | Normal | Normal | Normal | Normal | Normal |
| Day 0 pm | Normal | Normal | Normal | Normal | Normal |
| Day 1 am | Normal | freq cough | Normal | Normal | Normal |
| Day 1 pm | Normal | infreq cough | Normal | Normal | Normal |
| Day 2 am | Freq cough | serous nasal | Normal | Normal | Normal |
| Day 2 pm | serous nasal | Infreq cough, | sneezing | serous nasal | induced cough, |
| | | serous nasal | | | sneezing |
| Day 3 am | Normal | Normal | Normal | Normal | Normal |
| Day 3 pm | Freq cough | infreq cough | Normal | Normal | Normal |
| Day 4 am | Normal | Normal | infreq cough | Normal | Normal |
| Day 4 pm | infreq | Normal | Normal | Normal | Normal |
| | cough, | | | | |
| | sneezing | | | | |
| Day 5 am | Normal | Normal | Normal | Normal | Normal |

TABLE 2
RECTAL TEMPERATURES OF DOGS CHALLENGED WITH 6
LOGS OF EIV OHIO 03

| DPC | C5 1003 | C5 1005 | C5 1103 | C5 1106 | C5 1107 |
|---|---|---|---|---|---|
| Day −2 am | 102.1 | 102.0 | 102.6 | 102.4 | 102.7 |
| Day −2 pm | 102.1 | 102.6 | 102.5 | 102.6 | 103.0 |
| Day −1 am | 102.3 | 102.5 | 103.1 | 102.4 | 102.2 |
| Day −1 pm | 102.5 | 102.5 | 102.4 | 102.6 | 102.5 |
| Day 0 am | 102.3 | 102.5 | 102.4 | 102.3 | 103.2 |
| Day 0 pm | 101.0 | 101.7 | 101.1 | 100.8 | 101.3 |
| Baseline | 102.1 | 102.3 | 102.4 | 102.2 | 102.5 |
| Day 1 am | 101.4 | 102.0 | 102.2 | 102.2 | 101.7 |
| Day 1 pm | 101.7 | 101.6 | 102.2 | 102.1 | 101.9 |
| Day 2 am | 103.2 | 102.4 | 102.7 | 102.4 | 103.1 |
| Day 2 pm | 101.4 | 101.8 | 101.8 | 101.7 | 101.7 |
| Day 3 am | 102.4 | 103.4 | 101.9 | 102.8 | 101.8 |
| Day 3 pm | 102.3 | 102.3 | 102.2 | 102.4 | 102.3 |
| Day 4 am | 101.6 | 100.8 | 102.0 | 102.0 | 101.6 |
| Day 4 pm | 101.9 | 102.1 | 101.9 | 102.2 | 101.9 |
| Day 5 am | 102.2 | 102.0 | 102.4 | 102.5 | 102.3 |

TABLE 3
RESPIRATORY SIGNS DETECTED IN DOGS CHALLENGED WITH 8 LOGS
OF EIV OHIO 03

| DPC | C5 1001 | C5 1002 | C5 1004 | C5 1105 | C5 1108 |
|---|---|---|---|---|---|
| Day −2 am | Normal | Normal | Normal | Normal | Normal |
| Day −2 pm | Normal | Normal | Normal | Normal | Normal |
| Day −1 am | Normal | Normal | Normal | Normal | Normal |
| Day −1 pm | Normal | Normal | Normal | Normal | Normal |
| Day 0 am | Normal | Normal | Normal | Normal | Normal |
| Day 0 pm | Normal | Normal | Normal | Normal | Normal |
| Day 1 am | serous nasal | Normal | Normal | Normal | Normal |
| Day 1 pm | | Normal | Normal | Normal | Normal |
| Day 2 am | serous nasal | Normal | Normal | Normal | infreq cough |
| Day 2 pm | Normal | Normal | Normal | Normal | Normal |
| Day 3 am | Normal | Normal | mild muco ocular | Normal | Normal |
| Day 3 pm | Normal | Normal | Normal | Normal | Normal |
| Day 4 am | Normal | Normal | Normal | Normal | Normal |
| Day 4 pm | Freq cough | Normal | Normal | Normal | Normal |
| Day 5 am | Normal | Normal | Normal | Normal | Normal |

TABLE 4
RECTAL TEMPERATURES OF DOGS CHALLENGED WITH 8
LOGS OF EIV OHIO 03

| DPC | C5 1001 | C5 1002 | C5 1004 | C5 1105 | C5 1108 |
|---|---|---|---|---|---|
| Day −2 am | 101.1 | 101.4 | 102.4 | 101.9 | 102.6 |
| Day −2 pm | 101.3 | 101.6 | 102.2 | 102.1 | 101.0 |
| Day −1 am | 101.9 | 102.1 | 102.0 | 102.0 | 102.0 |
| Day −1 pm | 102.5 | 102.8 | 102.4 | 102.6 | 102.5 |
| Day 0 am | 102.3 | 101.8 | 102.5 | 102.2 | 102.4 |
| Day 0 pm | 100.2 | 101.4 | 101.8 | 101.5 | 102.1 |
| Baseline | 101.6 | 101.9 | 102.2 | 102.1 | 102.1 |
| Day 1 am | 101.4 | 102.3 | 102.2 | 101.9 | 102.6 |
| Day 1 pm | 103.1 | 103.2 | 102.5 | 102.0 | 102.7 |
| Day 2 am | 102.2 | 101.6 | 101.9 | 101.6 | 102.6 |
| Day 2 pm | 102.5 | 102.1 | 102.3 | 102.5 | 102.6 |
| Day 3 am | 102.2 | 102.5 | 103.2 | 103.0 | 103.1 |
| Day 3 pm | 101.5 | 101.7 | 101.7 | 102.0 | 101.7 |
| Day 4 am | 101.2 | 101.9 | 102.1 | 101.7 | 102.0 |
| Day 4 pm | 102.4 | 102.6 | 102.6 | 101.9 | 102.4 |
| Day 5 am | 101.7 | 101.2 | 102.0 | 101.5 | 101.8 |

TABLE 5
VIRAL ISOLATION FROM DOGS CHALLENGED WITH 6 OR 8 LOGS OF EIV OHIO 03

| | | | | | | | Bronchial | Pharyngeal | |
|---|---|---|---|---|---|---|---|---|---|
| | | | | | | Retropharyngeal | Lymph | Swabs | |
| Group | Dog ID | Trachea | Lung | Thymus | Tonsil | Node | Node | Day −1 | Day 0 |
| 1 (8 | C5- | A | 0 | 0 | 0 | 0 | 0 | 0 | 0 |
| logs) | 1001 | | | | | | | | |
| | C5- | A | 0 | 0 | 0 | 0 | 0 | 0 | 0 |
| | 1002 | | | | | | | | |
| | C5- | 0 | 0 | 0 | 0 | 0 | 0 | 0 | 0 |
| | 1004 | | | | | | | | |
| | C5- | 0 | 0 | 0 | 0 | 0 | 0 | 0 | 0 |
| | 1105 | | | | | | | | |
| | C5- | A | A | 0 | 0 | 0 | 0 | 0 | 0 |
| | 1108 | | | | | | | | |
| 2 (6 | C5- | A | 0 | 0 | A | 0 | 0 | 0 | 0 |
| logs) | 1003 | | | | | | | | |
| | C5- | A | A | 0 | A | 0 | 0 | 0 | 0 |
| | 1005 | | | | | | | | |
| | C5- | A | 0 | 0 | 0 | 0 | 0 | 0 | 0 |
| | 1103 | | | | | | | | |
| | C5- | A | A | 0 | A | 0 | 0 | 0 | 0 |
| | 1106 | | | | | | | | |
| | C5- | A | A | A | A | 0 | 0 | 0 | 0 |
| | 1107 | | | | | | | | |

| | | Pharyngeal | | | | | Nasal | |
|---|---|---|---|---|---|---|---|---|
| | | Swabs | | | | | Swabs | |
| Group | Dog ID | Day 1 | Day 2 | Day 3 | Day 4 | Day 5 | Day 2 | Day 3 |
| 1 (8 | C5- | 0 | 0 | 0 | 0 | 0 | A | A |
| logs) | 1001 | | | | | | | |
| | C5- | 0 | 0 | 0 | 0 | 0 | A | A |
| | 1002 | | | | | | | |
| | C5- | 0 | 0 | 0 | 0 | 0 | A | A |
| | 1004 | | | | | | | |
| | C5- | A | 0 | 0 | 0 | 0 | A | A |
| | 1105 | | | | | | | |
| | C5- | 0 | 0 | 0 | 0 | 0 | A | A |
| | 1108 | | | | | | | |
| 2 (6 | C5- | 0 | A | 0 | 0 | 0 | A | A |
| logs) | 1003 | | | | | | | |
| | C5- | 0 | A | 0 | 0 | A | A | A |
| | 1005 | | | | | | | |
| | C5- | A | 0 | 0 | 0 | A | 0 | A |
| | 1103 | | | | | | | |
| | C5- | 0 | A | 0 | 0 | 0 | A | A |
| | 1106 | | | | | | | |
| | C5- | 0 | A | 0 | 0 | 0 | A | A |
| | 1107 | | | | | | | |

A = positive for viral isolation
0 = negative for viral isolation

### EXAMPLE 2

### Efficacy of a Killed Influenza Vaccine Against an Experimental Challenge with Canine Influenza Virus

Animals

Animals sero-negative to EIV Kentucky 97 were included in this study. Thirty-two (32) dogs of Beagle or Mongrel breed from 5 litters were assigned to two study groups using a computer generated randomization program. Each animal received a computer generated random number using Microsoft Excel. The animals were then sorted by litter followed by random number in ascending order. The animals were randomized into two test groups: one vaccinated group of 21 animals and one unvaccinated control group of 11 animals.

Vaccine

Standard methods were used to make the vaccine. Briefly, the EIV Kentucky 97 antigen used in blending the test vaccine was blended at 1500 hemagglutination (HA) units per dose at TT/PI along with a co-polymer adjuvant.

Vaccination

Dogs were 6 to 7 weeks old at the time of the first vaccination. Dogs in the vaccinated group were vaccinated subcutaneously twice, three weeks apart, with the test vaccine at 1500 hemagglutination (HA) units/dose. The two vaccinations were administered as a 1 ml dose and were administered on opposite sides of the neck.

Challenge

Canine Influenza Virus New York 05 (A/canine/NY/9/05) was obtained from Dr. Edward Dubovi at Cornell University. The virus was subcultured once in SPF eggs for establishment of an adequate volume of challenge material. The challenge virus was stored at −80° C. prior to use. On the day of challenge, two weeks after the second vaccination, the challenge virus was thawed quickly and diluted in order to obtain the targeted dose of 10^{6.5} EID₅₀. Aliquots of the challenge virus were kept on ice throughout the challenge procedure. Each dog was challenged intranasally with an aliquot of virus (2 ml) using a nebulizer. To facilitate the challenge, the dogs were sedated according to standard methods. Briefly, Robinul-V® was given at 5 μg/lb body weight intramuscularly followed by intramuscular administration of Telazol® at 7 mg/lb body weight approximately 15 minutes later.

To establish baselines, rectal temperatures were monitored and dogs were observed for coughing, nasal discharge, sneezing, and ocular discharge twice daily for two days prior to challenge (−2 DPC) and once in the morning of 0 DPC. “DPC” as used herein means days post challenge. Discharge was classified as mild, moderate, or severe. Respiratory signs and rectal temperatures were also observed and monitored twice daily thereafter until 7 DPC. Nasal swabs and pharyngeal swabs were collected daily for detection of viral shedding starting −1 DPC until 7 DPC for all the dogs.

Dogs were bled for serum on the day of the first vaccination (0 DPV1), 0 DPV2 (21 DPV1), 13 DPV2 and 8 DPC (the day of necropsy). “DPV” as used herein means days post vaccination. Nasal and pharyngeal swabs were collected daily for virus isolation from each dog starting 1 day prior to challenge until 7 DPC. All swabs collected were placed in sample tubes containing 3 ml of transport media (PBS/Glycerol with 2× gentamicin) and stored at −80° C. until testing.

All the dogs were euthanized and necropsied at 8 DPC. Lungs, trachea, tonsils, retropharyngeal lymph nodes, and bronchial lymph nodes were examined for significant gross pathology. Tissue samples (e.g., lung, trachea, tonsil, and lymph nodes) were collected for histopathological examination. Virus isolation from lung, trachea, and tonsil samples was attempted.

Sample Testing

Serum samples were tested by hemagglutination inhibition (HAI) assay for antibody titers to CIV New York 05. The assay used 8 HA units of the test indicator virus. All serum samples were pretreated with periodate and heat inactivated to remove any non-specific inhibitors. Virus shedding was detected by performing virus isolation from nasal swabs. In addition, virus isolation from pharyngeal swabs was performed. Swabs collected were thawed and the tubes were mixed by vortexing. Liquid was extracted from the swabs and the materials were tested using embryonated eggs. Briefly, 100 μl of sample was inoculated into 9-11 day old embryonated eggs. The eggs were allowed to incubate at 36±2° C. for 72 hours with daily observations for embryo death. Eggs that died within the first 24 hours were discarded. Eggs that died after the first 24 hours were tested for HA activity. At 72 hours post inoculation all remaining eggs were placed at 4° C. overnight, harvested and tested for HA activity.

The primary outcome was initially defined as the occurrence of virus shedding, as detected by virus isolation from nasal or pharyngeal swabs. The occurrence of clinical signs and fever post-challenge were initially defined as secondary outcomes.

Data Analysis—Estimator

The estimator was the vaccine efficacy (VE) statistic. Vaccine efficacy was calculated as the complement of the risk ratio:
VE=1−pᵥ/p_{c}
where pᵥ is the proportion of dogs with positive virus isolation in the vaccinated group and p_{c} is the proportion of dogs with positive virus isolation in the control group. The vaccine efficacy statistic was calculated for isolation from both nasal and pharyngeal isolations.

Data Analysis—Hypothesis Statement

This study was originally intended to test the null hypothesis that there is no difference in the proportion of dogs with positive virus isolation between the vaccinated group and the control group.
H_{O}: pᵥ=p_{c}
H_{A}: pᵥ≢p_{c}
where pᵥ=the proportion of dogs with positive virus isolation in the vaccinated group and p_{c}=the proportion of dogs with virus isolation in the control group.

Data Analysis—Statistical Analysis

Baseline assessment: The frequency distributions of the continuous outcome variables were assessed using PROC UNIVARIATE. Antibody titres were log transformed. Baseline evaluations to evaluate comparability of groups for litter, sex, and room were made by chi-square. A baseline evaluation to evaluate allocation of litters to rooms was made by chi-square.

Statistical methods: The number of dogs with positive virus isolation from nasal and pharyngeal secretions was compared between groups by Fisher's Exact test. Secondary outcomes were assessed for clinical signs, fever, antibody titer, and isolation from tissues. For the evaluation of clinical signs, an animal with any abnormal signs was categorized as positive for clinical signs. The proportion of days with positive clinical signs, calculated as the number of observations of positive clinical signs as a proportion of the number of observations, was compared between groups by Wilcoxon Rank Sum with the proportion of days with positive clinical signs as the dependent variable and treatment included as an independent variable with DAM included as a covariate. Also, the least square means and their 95% confidence intervals were constructed. This analysis was repeated for four individual clinical signs: coughing, sneezing, serous nasal discharge, and mucoid nasal discharge. No adjustments were made for multiple study endpoints.

The occurrence of these four signs (coughing, sneezing, serous nasal discharge, and mucoid nasal discharge) was further assessed by comparing the number of animals with each clinical sign in the vaccinated group to the number of animals with each clinical sign in the control group by Fisher's Exact test.

For the evaluation of fever, a mean rectal temperature baseline for each animal was calculated as the average of the temperature during the time before challenge. The difference between post-challenge temperature and baseline temperature was calculated to assess fever. Fever was compared between groups in a repeated measures analysis of variance (ANOVA) model with fever as the dependent variable and treatment, time, and the treatment*time interaction included as independent variables. The baseline rectal temperature was included as a covariate in the model and the DAM was included as a random effect covariate.

For the evaluation of antibody titers, the post-challenge antibody titers were compared between treatment groups in an analysis of variance (ANOVA) model with post-challenge antibody titer as the dependent variable and treatment, time, and the treatment*time interaction included as independent variables. The DAM was included as a random effect covariate.

For the evaluation of isolation from tissues, a frequency table, stratified by treatment group, was constructed for the occurrence of positive isolation from tonsil. There were no positive isolations from trachea or lung in either group. No further statistical analysis was performed due to the low recovery rate from both groups on tissue isolation.

All statistical analysis was performed using the SAS system (SAS Institute, Inc.). The level of significance was set at p<0.05.

Data Analysis—Assessment of Bias

Group (vaccinates or controls) assignments were made randomly. Personnel who conducted animal observations and laboratory measurements were blinded to treatment assignment. Therefore, any measurement bias should have affected both treatment groups equally, e.g., non-differential misclassification bias. Thus, any systematic information or measurement bias should be minimal and would expectedly cause a bias toward “no effect” or “no association”. The random assignment of study subjects to the treatment groups should have minimized sources of selection bias, which, if they existed, would also have been non-differential misclassification bias.

Three dogs in the vaccinated group and four dogs in the control group were reported with mild ocular discharge prior to initiation of the challenge portion of the study. Two dogs (one vaccinate and one control) were diagnosed with a prolapsed third eyelid during the observation period of the study. Therefore, the source of ocular discharge (challenge or prolapsed third eyelid) recorded after challenge may not have been specific to the challenge but may have been due to this pre-existing condition. However, since these existing conditions affected both treatment groups approximately equally and since observers were blinded as to treatment group, these pre-existing conditions should not have introduced substantial measurement bias. While, it is possible that some of the clinical signs observed after challenge may have reflected amplification of pre-existing clinical signs seen before challenge, this still should have affected both groups equally due to the equal distribution of these animals within both groups. When the data from the two dogs with prolapsed third eyelid are excluded from the analysis, the overall conclusion does not change regarding the efficacy of the test vaccine.

Challenge Virus Titers

Due to the number of dogs being challenged, two teams of personnel conducted the challenge concurrently. Samples of the challenge virus were collected immediately before challenge and immediately after challenge for retrospective titration. The challenge material from the first team ran out and was replenished with the aforementioned remaining challenge material. Four dogs (three vaccinates and one control) were challenged with this virus material.

The retrospective titers of aliquots of the actual challenge material were very close to the target titer of 10^{6.5} TCID₅₀/dose and the differences among the aliquots were minimal and within experimental error. Due to the shortage of dogs from both external and internal sources, a titration study to determine the challenge does of CIV was not conducted when the challenge virus was acquired from Cornell University. In addition, there was no challenge data generated elsewhere using CIV New York 05. The decision of choosing 10^{6.5} EID₅₀/dose was based on our previous experience with EIV Ohio 03 in dogs.

Clinical Observations

After challenge, seven (7) out of eleven (11) control dogs (64%) were observed with coughing while only three (3) out of twenty-one (21) vaccinates (14%) coughed (see Table 6 and Table 9). Furthermore, control dogs were more severely affected since coughing was observed on multiple days while each of the affected vaccinates was observed coughing only once. Some of the coughs observed were characterized as non-productive dry, hacking, or gagging coughs. Coughing has been identified as the most prominent respiratory sign observed in the CIV outbreaks.

Another respiratory sign commonly observed in the dogs during outbreaks is mucopurulent nasal discharge. Six (6) control dogs (55%) but none (0%) of the vaccinated dogs were observed with nasal mucoid discharge (see Table 6 and Table 9). The observer did not record this respiratory sign as mild mucopurulent discharge because the color of the discharge was “yellowish” but not “greenish”. However, the description of this respiratory sign provided by the observer actually matches with a mild form of mucopurulent nasal discharge.

In addition to mucopurulent nasal discharge, mild nasal serous discharge was observed in both controls and vaccinated dogs, although 73% of control dogs were affected as compared to 38% of vaccinated dogs (see Table 6 and Table 9). In the published literature, serous nasal discharge in clinically affected dogs during CIV outbreaks was rarely or never mentioned. It is not clear whether or not this respiratory sign was presented with the clinically affected dogs but was considered as insignificant.

Similar to serous nasal discharge, sneezing was observed in both control and vaccinated dogs. A larger percentage of control dogs (73%) were affected with this respiratory sign as compared to vaccinates (62%) (see Table 6 and Table 9).

Ocular discharge was also one of the clinical signs observed in this study and in previous studies using EIV Ohio 03. This clinical sign has never been mentioned in the clinically affected dogs during the outbreaks. In addition, dogs at young ages are prone to have mild serous ocular discharge due to non-specific causes. In fact, there were two dogs with a prolapsed third eyelid that was unrelated to the challenge (see Table 9). In any event, mild serous ocular discharge was observed in a few animals prior to challenge and in a few more animals after challenge. The relevance of this sign in association with CIV infection is questionable.

Low grade fever (≧103° F. but <103.5° F. and 1° F. above baseline) was detected in every control animal at least once after the challenge while none of the vaccinates had low grade fever (≧103.5° F. and 1° F. above baseline) or fever. The definitions of low grade fever and fever are consistent with those used in previous studies.

In particular, control animal C5 2804 had a low grade fever of 103.0° F. on 5DPC AM and 5DPC PM. Control animal C5 2806 had a low grade fever of 103.0° F. on 7DPC PM. Control animal C5 2902 had a low grade fever of 103.3° F. on 2DPC PM and a low grade fever of 103.1° F. on 5DPC PM. Control animal C5 3001 had a low grade fever of 103.2° F. on 2DPC AM and 2DPC PM and a low grade fever of 103.1° F. on 5DPC PM. Control animal C5 3005 had a low grade fever of 103.0° F. on 2DPC AM. Control animal C5 3102 had a low grade fever of 103.2° F. on 4DPC PM. Control animal C5 3102 was also had a fever on two different days (a fever of 104.1° F. on 2DPC AM; 104.0° F. on 2DPC PM, and a fever of 103.8° F. on 5DPC AM). Control animal C5 3103 had a low grade fever of 103.1° F. and 103.0° F. on 2DPC AM and 2DPC PM, respectively. Control animal C5 3106 had a low grade fever of 103.4° F. on 2DPC PM. Control animal C5 3205 had a low grade fever of 103.1° F. on 5DPC AM. Control animal C5 3206 had a low grade fever of 103.2° F. on 6DPC AM. Control animal C5 3208 had a low grade fever of 103.0° F. on 4DPC AM, 5DPC AM, and 5DPC PM.

The mean maximum body temperature was 102.5° F. (95% Cl 102.3, 102.7) and 103.2° F. (95% Cl 103.0, 103.4) for vaccinated and control dogs respectively. The mean difference from baseline for body temperature was higher in control dogs compared to vaccinates by an estimated 0.40 degrees F. (SE 0.05, 95% Cl 0.27, 0.53).

Results of clinical observations were inadvertently not recorded for one animal at −1 DPC (AM) and for three animals at 5 DPC (AM). All were vaccinates. According to one observer, those animals had no clinical signs but those observations were not recorded immediately after observation and the documentation error was missed. This missing clinical observation data, in our opinion, did not change the overall picture of the challenge results.

Due to the lack of knowledge of the ability of CIV New York 05 to induce clinical disease and the fact that, at least in one study, CIV Florida 04 did not induce clinical disease in dogs after experimental challenge (Crawford, P C, Dubovi, E J, Cattleman, W L, et al. 2005 Science 310:482-485), no clinical case definition was defined in the protocol for this study. In this study, 17 out of 21 vaccinated dogs and 11 out of 11 control dogs had at least a single occurrence of positive clinical signs (e.g., coughing, nasal discharge, ocular discharge, and sneezing). There was no difference between groups for the occurrence of any clinical signs. There were significant reductions in the proportion of observations with positive clinical signs. The attributable rate for vaccination (difference in proportion of days with positive clinical signs) for the number of observations of positive clinical signs was 28.1% (SE 0.11, 95% Cl −0.03, 59.4). The mitigated fraction for the reduction in the number of days with positive clinical signs was 51.9% (95% Cl 18.2, 85.7). The vaccinated dogs had positive clinical signs an estimated 2.26 fewer days (SE 0.84, 95% Cl −0.19, 4.50) compared to controls.

The vaccine significantly protected the dogs against coughing and mucopurulent nasal discharge which are the most common respiratory signs associated with CIV infection in dogs. The clinical disease induced by the experimental challenge mimics the most prevalent form of the clinical disease caused by CIV as observed in the outbreaks. The use of SPF eggs instead of cell culture for cultivating challenge virus and the use of a device to generate aerosol to deliver the challenge inoculum instead of instilling the inoculum without aerosolization are very likely associated with the success of inducing typical clinical signs in the challenged dogs in our study while others failed to do so in a previous study (see Crawford, P C, Dubovi, E J, Cattleman, W L, et al. 2005 Science 310:482-485) using CIV Florida 04.

Viral Isolation

Similar to the results from the three recent studies using EIV Ohio 03, 100% of control dogs shed virus based on the virus isolation results of nasal swabs (see Table 7 and Table 10). In contrast, only four (4) vaccinates (19%) had positive isolation. The vaccine efficacy against shedding was 80.9% (95% Cl 58.1, 94.6). In addition, control animals shed more days than the vaccinated animals (see Table 7 and Table 10).

After challenge, CIV was also isolated from pharyngeal swabs in all except one control dog (91%) while positive virus isolation was detected in only five vaccinates (24%) (see Table 7 and Table 11). The vaccine efficacy against shedding was 73.8% (95% Cl 42.4, 90.5). Again, control animals shed more days than the vaccinated animals (see Table 7 and Table 11).

Other than tonsils of a few vaccinates, no virus was isolated from any other tissues collected from any animals during necropsy (see Table 7 and Table 12). This finding is different from those of the studies with EIV Ohio 03 when dogs were usually necropsied at 5 DPC. In the study using CIV Florida 04 (see Crawford, P C, Dubovi, E J, Cattleman, W L, et al. 2005 Science 310:482-485), it was reported that no virus was isolated from the challenged dogs when necropsy was conducted 14 days after the challenge although positive isolation of CIV was detected in one of the two challenged dogs necropsied 5 days after challenge. It is possible that the scanty isolation rate of tissue samples in this study might be related to the fact that these dogs were necropsied at 8 DPC instead of 5 DPC.

Serological Response

Serological responses against CIV New York 05 as measured by HAI assay indicate a significant sero-conversion (4 fold or more increase in titer) after two vaccinations (see Table 8 and Table 13). It is a well known fact that humoral immunity plays an important protective role in disease caused by influenza viruses. Therefore, the induction of high antibody titers against CIV by the test vaccine provides additional evidence for the efficacy of the test vaccine against CIV.

Based on the antibody response in the controls, CIV New York 05 is very immunogenic in dogs since all control dogs sero-converted at 8 DPC after “intranasal” challenge (see Table 13).

Microscopic Examination of Tissue Samples Collected During Necropsy

Some gross lesions were observed in the lungs examined during necropsy. Fixed samples from lung, trachea, tonsil, and lymph nodes were submitted to Cornell University from microscopic examination by Dr. Brad Njaa. According to the results, samples from all control animals except one (C5 3106) were observed with tracheitis, bronchitis, and bronchiolitis in varying degree of severity. Tracheitis was detected in C5 3106. Interstitial pneumonia was detected in 7 control animals (C5 2804, C5 2806, C5 2902, C5 3005, C5 3205, C5 3206, and C5 3208). In contrast, no microscopic lesions were detected in the lung and trachea samples from any of the vaccinates whether or not they were observed with any of the clinical signs. This would indicate a much milder clinical disease for those vaccinates with any clinical signs as compared to the controls.

Results from this study, using CIV New York 05 as the challenge virus, demonstrate that a killed vaccine containing EIV Kentucky 97 at 1,500 HA/dose is efficacious against viral shedding in dogs after challenge with Canine Influenza Virus. Clinical signs were defined as secondary outcomes in the protocol due to the lack of knowledge whether or not clinical disease can be induced by CIV New York 05. However, results based on clinical observation of respiratory signs and microscopic examination of lung and trachea samples unequivocally demonstrate the efficacy of the vaccine against clinical disease associated with CIV infection. Therefore, results from this study support the label claim of the vaccine “For vaccination of healthy dogs eight weeks of age or older as an aid in the prevention of viral shedding and disease caused by canine influenza virus.”

TABLE 6
RESPIRATORY SIGNS OBSERVED IN VACCINATED AND NON-
VACCINATED CONTROL DOGS CHALLENGED WITH CANINE
INFLUENZA VIRUS (CIV) NEW YORK 05

| | | | Nasal Mucoid | | Serous Nasal | | | |
|---|---|---|---|---|---|---|---|---|
| | Coughing^{b} | | Discharge | | Discharge^{c} | | Sneezing^{d} | |
| | % of | Incidence | % of | Incidence | % of | Incidence | % of | Incidence |
| Group^{a} | Dogs | Intensity^{e} | Dogs | Intensity | Dogs | Intensity | Dogs | Intensity |
| 1 (n = 21) | 14* | 0.01 | 0* | 0* | 38 | 0.048 | 62 | 0.065 |
| 2 (n = 11) | 64 | 0.30 | 55 | 0.06 | 73 | 0.071 | 73 | 0.104 |

^{a}Group 1: dogs vaccinated with a vaccine containing 1,500 hemagglutinin (HA) units of EIV Kentucky 97; Group 2: unvaccinated controls.
^{b}All spontaneous.
^{c}Only mild serous nasal discharge was observed.1
^{d}Including sneezing after swabbing.
^{e}Number of positive episodes observed divided by the total number of observations.
*The value is significantly different from that of the corresponding control group, p < 0.05.

TABLE 7
VIRAL ISOLATION FROM VACCINATED AND NON-VACCINATED
CONTROL DOGS CHALLENGED WITH CANINE INFLUENZA VIRUS
(CIV) NEW YORK 05

| | | | Pharyngeal | | | | |
|---|---|---|---|---|---|---|---|
| | Nasal Swab | | Swab | | Lung | Trachea | Tonsil |
| | | Incidence | | Incidence | % of | % of | % of |
| Group^{a} | % of Dogs | Intensity^{b} | % of Dogs | Intensity | Dogs | Dogs | Dogs |
| 1 (n = 21) | 19* | 0.57 | 24* | 0.86 | 0 | 0 | 19 |
| 2 (n = 11) | 100 | 8.00 | 91 | 3.57 | 0 | 0 | 0 |

^{a}Group 1: dogs vaccinated with a vaccine containing 1,500 hemagglutinin (HA) units of EIV Kentucky 97; Group 2: unvaccinated controls.
^{b}Number of days of positive detection divided by the total number of days.
*The value is significantly different from that of the corresponding control group, p < 0.05.

TABLE 8
SEROLOGICAL RESPONSE^{A} AGAINST CANINE INFLUENZA
VIRUS (CIV) NEW YORK 05 MEASURED BY
HEMAGGLUTINATION INHIBITION (HAI) ASSAY IN DOGS
VACCINATED WITH A KILLED VACCINE CONTAINING
EQUINE INFLUENZA VIRUS (EIV)
KENTUCKY 97 AND UNVACCINATED CONTROLS

| Group^{b} | 0 DPV1^{c} | 0 DPV2 | 13 DPV2 | 8 DPC^{d} |
|---|---|---|---|---|
| 1 (n = 21) | 4 ± 0 | 23 ± 2 | 356 ± 2 | 959 ± 2* |
| 2 (n = 11) | 4 ± 0 | 4 ± 0 | 4 ± 0 | 88 ± 2 |

^{a}Results are expressed as mean geometric mean titer (GMT) ± standard deviation. To facilitate the calculation of GMT, an HAI titer of <8 is considered as 4 and a titer > 1024 is considered as 2048.
^{b}Group 1 dogs were vaccinated with a vaccine containing 1,500 HA units of EIV Kentucky 97; Group 2 dogs served as unvaccinated controls.
^{c}DPV: Days post vaccination
^{d}DPC: Days post challenge
*The value is significantly different from that of the corresponding control group, p < 0.05.

TABLE 9
Clinical Observations of Vaccinated and Non-vaccinated Dogs Challenged with Canine Influenza Virus (CIV)

| | | −2DPC^{b} | −2DPC | −1DPC | −1DPC | 0DPC | 1DPC | 1DPC | 2DPC | 2DPC |
|---|---|---|---|---|---|---|---|---|---|---|
| Dog ID | Group^{a} | AM | PM | AM | PM | AM | AM | PM | AM | PM |
| C5 2801 | 1 | A | A | A | A | A | A | A | A | SN |
| C5 2802 | 1 | A | A | A | A | A | OS1 | A | A | A |
| C5 2803 | 1 | A | A | A | A | A | OS1, | OS1, O | O (EP, | O (EP, |
| | | | | | | | O (EP) | (OMP) | OMP) | OMP) |
| C5 2805 | 1 | A | A | A | A | A | NSI | A | OS1 | OS1 |
| C5 2901 | 1 | A | A | A | A | A | OS1 | A | A | A |
| C5 2903 | 1 | A | A | A | A | A | NS1 | A | A | A |
| C5 2904 | 1 | A | A | No Entry | A | A | A | A | A | A |
| C5 3002 | 1 | A | A | A | A | A | A | A | A | A |
| C5 3003 | 1 | A | A | A | A | A | A | A | A | A |
| C5 3004 | 1 | A | A | A | A | A | A | OS1 | A | A |
| C5 3006 | 1 | A | A | A | A | A | A | A | A | A |
| C5 3101 | 1 | A | A | A | A | A | A | A | A | A |
| C5 3104 | 1 | A | A | A | A | A | A | A | A | A |
| C5 3105 | 1 | A | OS1 | OS1 | A | A | OS1 | A | OS1 | OS1 |
| C5 3107 | 1 | A | A | A | A | A | A | A | A | A |
| C5 3201 | 1 | A | A | A | A | A | A | A | A | A |
| C5 3202 | 1 | A | A | A | A | A | A | A | SN | A |
| | | | | | | | | | (FS) | |
| C5 3203 | 1 | A | A | A | A | A | A | NS1 | A | A |
| C5 3204 | 1 | A | A | A | A | A | A | A | SN | A |
| | | | | | | | | | (FS) | |
| C5 3207 | 1 | A | A | A | A | A | A | A | A | A |
| C5 3209 | 1 | OS1 | A | OS1 | OS1 | OS1 | A | OS1 | OS1 | A |
| C5 2804 | 2 | A | A | A | A | A | A | OS1 | OS1 | OS1, |
| | | | | | | | | | | NS1 |
| C5 2806 | 2 | A | A | A | O | A | A | A | OS1 | OS1 |
| | | | | | (Gant) | | | | | |
| C5 2902 | 2 | A | A | O | A | A | A | A | OS1 | OS1 |
| | | | | (Vomiting) | | | | | | |
| C5 3001 | 2 | A | A | A | A | A | A | A | A | A |
| C5 3005 | 2 | A | A | A | A | A | A | A | A | NS1 |
| C5 3102 | 2 | A | A | A | A | A | A | A | A | A |
| C5 3103 | 2 | OM1 | A | A | A | A | NS1, | A | A | A |
| | | | | | | | O (EP, | | | |
| | | | | | | | OMP) | | | |
| C5 3106 | 2 | A | A | A | A | A | OS1 | OS1 | OS1 | OS1 |
| C5 3205 | 2 | OS1 | OS1 | OS1 | OS1 | A | A | OS1 | A | A |
| C5 3206 | 2 | A | A | A | A | A | A | NS1 | A | A |
| C5 3208 | 2 | A | A | A | A | A | SN | A | SN | A |
| | | | | | | | | | (FS) | |

| | | 3DPC | 3DPC | 4DPC | 4DPC | 5DPC | 5DPC | 6DPC | 6DPC | 7DPC | 7DPC |
|---|---|---|---|---|---|---|---|---|---|---|---|
| Dog ID | Group^{a} | AM | PM | AM | PM | AM | PM | AM | PM | AM | PM |
| C5 | 1 | C2-A | A | A | A | A | A | A | A | A | A |
| 2801 | | | | | | | | | | | |
| C5 | 1 | NS1 | OS1 | A | SN, | A | A | A | A | A | A |
| 2802 | | | | | C2, | | | | | | |
| | | | | | OS1 | | | | | | |
| C5 | 1 | O | O | O | O | SN, O | SN, O | O (EP- | NS1, | SN, O | O |
| 2803 | | (EP, SD1- | (EP, SD1- | (EP, SD1- | (EP, SD1- | (EP, | (EP, | OMP) | O (EP- | (EP- | (EP- |
| | | P) | P) | P) | P) | SD1-P) | SD1- | | OMP) | OMP) | OMP) |
| | | | | | | | P) | | | | |
| C5 | 1 | SN, | OS1, | NS1 | OS1 | No | OS1 | OS1 | A | OS1 | A |
| 2805 | | NS1 | NS1 | | | Entry | | | | | |
| C5 | 1 | A | A | A | A | A | SN | A | SN | A | A |
| 2901 | | | | | | | | | | | |
| C5 | 1 | A | A | A | A | A | A | A | A | A | A |
| 2903 | | | | | | | | | | | |
| C5 | 1 | A | A | A | SN | A | A | A | A | A | A |
| 2904 | | | | | | | | | | | |
| C5 | 1 | A | A | A | A | A | A | A | A | A | A |
| 3002 | | | | | | | | | | | |
| C5 | 1 | A | NS1 | A | NS1 | No | SN | A | A | A | A |
| 3003 | | | | | | Entry | | | | | |
| C5 | 1 | A | A | A | A | A | A | A | A | A | A |
| 3004 | | | | | | | | | | | |
| C5 | 1 | A | A | A | A | No | C3-F | SN | A | A | A |
| 3006 | | | | | | Entry | | (FS) | | | |
| C5 | 1 | A | A | A | A | SN, | SN | A | A | A | A |
| 3101 | | | | | | OS1 | | | | | |
| C5 | 1 | SN | A | A | A | SN | A | A | A | SN | A |
| 3104 | | | | | | (FS) | | | | (FS) | |
| C5 | 1 | SN, | OS1 | A | NS1 | SN, | OS1 | OS1 | OS1 | OS1 | OS1, |
| 3105 | | OS1 | | | | OS1 | | | | | NS1 |
| C5 | 1 | A | A | A | A | A | A | A | A | A | A |
| 3107 | | | | | | | | | | | |
| C5 | 1 | A | A | A | A | A | A | A | A | A | A |
| 3201 | | | | | | | | | | | |
| C5 | 1 | A | A | A | A | A | A | A | A | A | A |
| 3202 | | | | | | | | | | | |
| C5 | 1 | A | A | A | NS1 | A | A | A | A | A | OS1 |
| 3203 | | | | | | | | | | | |
| C5 | 1 | A | A | NS1 | A | A | A | A | A | A | A |
| 3204 | | | | | | | | | | | |
| C5 | 1 | A | A | A | A | A | A | A | A | A | A |
| 3207 | | | | | | | | | | | |
| C5 | 1 | OS1 | OS1 | OS1 | A | OS1 | OS1 | A | OS1 | OS1 | OS1 |
| 3209 | | | | | | | | | | | |
| C5 | 2 | OS1 | OS1 | C2 | A | C3, | C3, | C3 | C3, | NMD | C3, |
| 2804 | | | | | | OS1 | NMD | | NMD | | OS1, |
| | | | | | | | | | | | NMD |
| C5 | 2 | A | A | A | A | A | A | NMD | A | A | SN, |
| 2806 | | | | | | | | | | | NMD |
| C5 | 2 | C3-B | A | SN, | SN, | SN, | C3 | C3 | C3 | C2, | C3, |
| 2902 | | | | C3-D, O | C2 | C3, | | | | NS1 | NMD |
| | | | | (sounds | | NS1 | | | | | |
| | | | | congested) | | | | | | | |
| C5 | 2 | A | A | NS1 | A | C3 | C2 | C3, | A | A | C2 |
| 3001 | | | | | | | | NMD | | | |
| C5 | 2 | A | A | A | A | NS1 | SN | A | NS1 | A | A |
| 3005 | | | | | | | | | | | |
| C5 | 2 | A | A | A | A | A | SN | A | A | A | A |
| 3102 | | | | | | | | | | | |
| C5 | 2 | OS1 | OS1 | SN, | A | SN, C3 | C3 | C3, | A | OS1 | NMD |
| 3103 | | | | C2-D, | | | | OS1 | | | |
| | | | | OS1, | | | | | | | |
| | | | | NS1 | | | | | | | |
| C5 | 2 | SN | SN | SN | A | A | SN | A | A | A | A |
| 3106 | | | | (FS- | | | | | | | |
| | | | | C) | | | | | | | |
| C5 | 2 | OS1 | A | OS1 | D, C3- | D, | D, C3- | NMD, | C3, | C3, | C3, |
| 3205 | | | | | E, | NS1, | D, | D, C3, | OS1, O | OS1 | OS1 |
| | | | | | OS1 | OS1, O | OS1, O | OS2 | (Dyspnea) | | |
| | | | | | | (Dyspnea) | (Dyspnea) | | | | |
| C5 | 2 | A | A | A | A | A | C3-B | D, C2, | D, C2, 0 | C2, 0 | C3 |
| 3206 | | | | | | | | O(Shivering) | (Dyspnea, | (Dyspnea) | |
| | | | | | | | | | Shivering) | | |
| C5 | 2 | C3-C | C3-A | D, C3-A | C3-E | C3-D, | C3-D, O | C3, | C3, | SN, | C3, |
| 3208 | | | | | | NS1 | (Dyspnea) | OS1 | OS1 | C2, | OS1 |
| | | | | | | | | | | OS1 | |

^{a}Group 1: dogs vaccinated with a vaccine containing 1500 hemagglutinin (HA) units of EIV Kentucky 97 Group 2: non-vaccinated controls
^{b}DPC: Days post challenge
A = Normal
D = Depressed
NS = Serous Nasal Discharge
1-mild, 2-moderate, 3-severe
OS = Serous Ocular Discharge
1-mild, 2-moderate, 3-severe
MN = Mucopurulent Nasal Discharge
1-mild, 2-moderate, 3-severe
OM = Mucopurulent Ocular Discharge
1-mild, 2-moderate, 3-severe
NMD = Mucoid Nasal Discharge
1-mild, 2-moderate, 3-severe
SN = Sneezing
FS—Following swabbing
FSC—Following swabbing but continued throughout observation period
C—Coughing
1-induced by palpation
2-infrequent (1-2 coughs per observation)
3-frequent (>2 coughs per observation)
A-Dry hacking cough
B-Nonproductive gagging cough
C-Nonproductive deep hacking cough
D-Dry nonproductive cough
E-Dry nonproductive hacking-gagging continuing throughout the observation period
F-Slight
O—Other
EP—3^{rd} Eyelid Prolapse
SDIP—Mild Serous discharge from prolapsed eye
OMP—Mucous discharge from prolapsed eye

TABLE 10
VIRAL ISOLATION FROM NASAL SWABS COLLECTED FROM VACCINATED
AND NON-VACCINATED DOGS CHALLENGED WITH CANINE INFLUENZA VIRUS
(CIV)

| Dog ID | Group^{a} | 0 DPC^{b} | 1 DPC | 2 DPC | 3 DPC | 4 DPC | 5 DPC | 6 DPC | 7 DPC |
|---|---|---|---|---|---|---|---|---|---|
| C5 | 1 | 0 | 0 | 0 | 0 | 0 | 0 | 0 | 0 |
| 2801 | | | | | | | | | |
| C5 | 1 | 0 | 0 | 0 | 0 | 0 | 0 | 0 | 0 |
| 2802 | | | | | | | | | |
| C5 | 1 | 0 | 0 | 0 | 0 | 0 | 0 | 0 | 0 |
| 2803 | | | | | | | | | |
| C5 | 1 | 0 | 0 | 0 | 0 | 0 | 0 | 0 | 0 |
| 2805 | | | | | | | | | |
| C5 | 1 | 0 | 0 | 0 | 0 | 0 | 0 | 0 | 0 |
| 2901 | | | | | | | | | |
| C5 | 1 | 0 | 0 | + | 0 | 0 | 0 | 0 | 0 |
| 2903 | | | | | | | | | |
| C5 | 1 | 0 | 0 | + | 0 | 0 | 0 | 0 | 0 |
| 2904 | | | | | | | | | |
| C5 | 1 | 0 | 0 | 0 | 0 | 0 | 0 | 0 | 0 |
| 3002 | | | | | | | | | |
| C5 | 1 | 0 | 0 | 0 | 0 | 0 | 0 | 0 | + |
| 3003 | | | | | | | | | |
| C5 | 1 | 0 | 0 | 0 | 0 | 0 | 0 | 0 | 0 |
| 3004 | | | | | | | | | |
| C5 | 1 | 0 | 0 | 0 | 0 | 0 | 0 | 0 | 0 |
| 3006 | | | | | | | | | |
| C5 | 1 | 0 | 0 | 0 | 0 | 0 | 0 | 0 | 0 |
| 3101 | | | | | | | | | |
| C5 | 1 | 0 | 0 | 0 | 0 | 0 | 0 | 0 | 0 |
| 3104 | | | | | | | | | |
| C5 | 1 | 0 | 0 | 0 | 0 | 0 | 0 | 0 | 0 |
| 3105 | | | | | | | | | |
| C5 | 1 | 0 | 0 | 0 | + | 0 | 0 | 0 | 0 |
| 3107 | | | | | | | | | |
| C5 | 1 | 0 | 0 | 0 | 0 | 0 | 0 | 0 | 0 |
| 3201 | | | | | | | | | |
| C5 | 1 | 0 | 0 | 0 | 0 | 0 | 0 | 0 | 0 |
| 3202 | | | | | | | | | |
| C5 | 1 | 0 | 0 | 0 | 0 | 0 | 0 | 0 | 0 |
| 3203 | | | | | | | | | |
| C5 | 1 | 0 | 0 | 0 | 0 | 0 | 0 | 0 | 0 |
| 3204 | | | | | | | | | |
| C5 | 1 | 0 | 0 | 0 | 0 | 0 | 0 | 0 | 0 |
| 3207 | | | | | | | | | |
| C5 | 1 | 0 | 0 | 0 | 0 | 0 | 0 | 0 | 0 |
| 3209 | | | | | | | | | |
| C5 | 2 | 0 | 0 | + | + | 0 | + | + | 0 |
| 2804 | | | | | | | | | |
| C5 | 2 | 0 | + | + | + | + | + | + | 0 |
| 2806 | | | | | | | | | |
| C5 | 2 | 0 | 0 | + | + | + | + | + | 0 |
| 2902 | | | | | | | | | |
| C5 | 2 | 0 | + | + | + | + | + | 0 | 0 |
| 3001 | | | | | | | | | |
| C5 | 2 | 0 | + | + | + | 0 | 0 | + | 0 |
| 3005 | | | | | | | | | |
| C5 | 2 | 0 | + | + | + | 0 | + | 0 | 0 |
| 3102 | | | | | | | | | |
| C5 | 2 | 0 | + | + | + | 0 | + | + | 0 |
| 3103 | | | | | | | | | |
| C5 | 2 | 0 | + | + | + | + | + | 0 | 0 |
| 3106 | | | | | | | | | |
| C5 | 2 | 0 | + | + | + | + | + | + | 0 |
| 3205 | | | | | | | | | |
| C5 | 2 | 0 | + | + | + | + | + | + | + |
| 3206 | | | | | | | | | |
| C5 | 2 | 0 | + | + | + | + | 0 | + | 0 |
| 3208 | | | | | | | | | |

^{a}Group 1: dogs vaccinated with a vaccine containing 1500 hemagglutinin (HA) units of EIV Kentucky 97 Group 2: non-vaccinated controls
^{b}DPC: Days post challenge
+ Indicates positive isolation
0 Indicates negative isolation

TABLE 11
VIRAL ISOLATION FROM PHARYNGEAL SWABS COLLECTED FROM
VACCINATED AND NON-VACCINATED DOGS CHALLENGED WITH CANINE
INFLUENZA VIRUS (CIV)

| Dog | | | | | | | | | 7 |
|---|---|---|---|---|---|---|---|---|---|
| ID | Group^{a} | 0 DPC^{b} | 1 DPC | 2 DPC | 3 DPC | 4 DPC | 5 DPC | 6 DPC | DPC |
| C5 | 1 | 0 | 0 | 0 | 0 | 0 | 0 | 0 | 0 |
| 2801 | | | | | | | | | |
| C5 | 1 | 0 | 0 | 0 | 0 | 0 | 0 | 0 | 0 |
| 2802 | | | | | | | | | |
| C5 | 1 | 0 | 0 | 0 | 0 | 0 | 0 | 0 | 0 |
| 2803 | | | | | | | | | |
| C5 | 1 | 0 | 0 | + | 0 | 0 | 0 | 0 | 0 |
| 2805 | | | | | | | | | |
| C5 | 1 | 0 | 0 | 0 | 0 | 0 | 0 | 0 | 0 |
| 2901 | | | | | | | | | |
| C5 | 1 | 0 | + | 0 | 0 | 0 | 0 | 0 | 0 |
| 2903 | | | | | | | | | |
| C5 | 1 | 0 | 0 | 0 | 0 | 0 | 0 | 0 | 0 |
| 2904 | | | | | | | | | |
| C5 | 1 | 0 | 0 | 0 | 0 | 0 | 0 | 0 | 0 |
| 3002 | | | | | | | | | |
| C5 | 1 | 0 | 0 | 0 | 0 | 0 | + | 0 | 0 |
| 3003 | | | | | | | | | |
| C5 | 1 | 0 | 0 | 0 | 0 | 0 | 0 | 0 | 0 |
| 3004 | | | | | | | | | |
| C5 | 1 | 0 | 0 | 0 | 0 | 0 | 0 | 0 | 0 |
| 3006 | | | | | | | | | |
| C5 | 1 | 0 | 0 | 0 | 0 | 0 | + | + | 0 |
| 3101 | | | | | | | | | |
| C5 | 1 | 0 | 0 | 0 | 0 | 0 | 0 | + | 0 |
| 3104 | | | | | | | | | |
| C5 | 1 | 0 | 0 | 0 | 0 | 0 | 0 | 0 | 0 |
| 3105 | | | | | | | | | |
| C5 | 1 | 0 | 0 | 0 | 0 | 0 | 0 | 0 | 0 |
| 3107 | | | | | | | | | |
| C5 | 1 | 0 | 0 | 0 | 0 | 0 | 0 | 0 | 0 |
| 3201 | | | | | | | | | |
| C5 | 1 | 0 | 0 | 0 | 0 | 0 | 0 | 0 | 0 |
| 3202 | | | | | | | | | |
| C5 | 1 | 0 | 0 | 0 | 0 | 0 | 0 | 0 | 0 |
| 3203 | | | | | | | | | |
| C5 | 1 | 0 | 0 | 0 | 0 | 0 | 0 | 0 | 0 |
| 3204 | | | | | | | | | |
| C5 | 1 | 0 | 0 | 0 | 0 | 0 | 0 | 0 | 0 |
| 3207 | | | | | | | | | |
| C5 | 1 | 0 | 0 | 0 | 0 | 0 | 0 | 0 | 0 |
| 3209 | | | | | | | | | |
| C5 | 2 | 0 | 0 | + | 0 | + | + | 0 | 0 |
| 2804 | | | | | | | | | |
| C5 | 2 | 0 | 0 | + | 0 | + | 0 | 0 | 0 |
| 2806 | | | | | | | | | |
| C5 | 2 | 0 | 0 | + | + | 0 | + | 0 | 0 |
| 2902 | | | | | | | | | |
| C5 | 2 | 0 | 0 | + | + | 0 | 0 | 0 | 0 |
| 3001 | | | | | | | | | |
| C5 | 2 | 0 | 0 | 0 | 0 | 0 | 0 | 0 | 0 |
| 3005 | | | | | | | | | |
| C5 | 2 | 0 | 0 | + | 0 | 0 | + | + | 0 |
| 3102 | | | | | | | | | |
| C5 | 2 | 0 | 0 | + | 0 | 0 | + | 0 | 0 |
| 3103 | | | | | | | | | |
| C5 | 2 | 0 | 0 | + | 0 | + | + | 0 | 0 |
| 3106 | | | | | | | | | |
| C5 | 2 | 0 | 0 | + | + | 0 | + | 0 | 0 |
| 3205 | | | | | | | | | |
| C5 | 2 | 0 | 0 | 0 | + | + | 0 | 0 | 0 |
| 3206 | | | | | | | | | |
| C5 | 2 | 0 | 0 | + | + | 0 | 0 | 0 | 0 |
| 3208 | | | | | | | | | |

^{a}Group 1: dogs vaccinated with a vaccine containing 1500 hemagglutinin (HA) units of EIV Kentucky 97 Group 2: non-vaccinated controls
^{b}DPC: Days post challenge
+ Indicates positive isolation
0 Indicates negative isolation

TABLE 12
VIRAL ISOLATION FROM TISSUE SAMPLES COLLECTED
FROM VACCINATED AND NON-VACCINATED DOGS
CHALLENGED WITH CANINE INFLUENZA VIRUS (CIV)

| Dog ID | Group^{a} | Trachea | Lung | Tonsil |
|---|---|---|---|---|
| C5 2801 | 1 | 0 | 0 | 0 |
| C5 2802 | 1 | 0 | 0 | 0 |
| C5 2803 | 1 | 0 | 0 | 0 |
| C5 2805 | 1 | 0 | 0 | + |
| C5 2901 | 1 | 0 | 0 | 0 |
| C5 2903 | 1 | 0 | 0 | + |
| C5 2904 | 1 | 0 | 0 | + |
| C5 3002 | 1 | 0 | 0 | 0 |
| C5 3003 | 1 | 0 | 0 | 0 |
| C5 3004 | 1 | 0 | 0 | 0 |
| C5 3006 | 1 | 0 | 0 | 0 |
| C5 3101 | 1 | 0 | 0 | 0 |
| C5 3104 | 1 | 0 | 0 | 0 |
| C5 3105 | 1 | 0 | 0 | + |
| C5 3107 | 1 | 0 | 0 | 0 |
| C5 3201 | 1 | 0 | 0 | 0 |
| C5 3202 | 1 | 0 | 0 | 0 |
| C5 3203 | 1 | 0 | 0 | 0 |
| C5 3204 | 1 | 0 | 0 | 0 |
| C5 3207 | 1 | 0 | 0 | 0 |
| C5 3209 | 1 | 0 | 0 | 0 |
| C5 2804 | 2 | 0 | 0 | 0 |
| C5 2806 | 2 | 0 | 0 | 0 |
| C5 2902 | 2 | 0 | 0 | 0 |
| C5 3001 | 2 | 0 | 0 | 0 |
| C5 3005 | 2 | 0 | 0 | 0 |
| C5 3102 | 2 | 0 | 0 | 0 |
| C5 3103 | 2 | 0 | 0 | 0 |
| C5 3106 | 2 | 0 | 0 | 0 |
| C5 3205 | 2 | 0 | 0 | 0 |
| C5 3206 | 2 | 0 | 0 | 0 |
| C5 3208 | 2 | 0 | 0 | 0 |

^{a}Group 1: dogs vaccinated with a vaccine containing 1500 hemagglutinin (HA) units of EIV Kentucky 97
Group 2: non-vaccinated controls
+ Indicates positive isolation
0 Indicates negative isolation

TABLE 13
SEROLOGICAL RESPONSE AGAINST CANINE INFLUENZA
VIRUS ANTIGEN MEASURED BY HEMAGGLUTINATION
INHIBITION ASSAY IN DOGS VACCINATED WITH A KILLED
VACCINE CONTAINING EQUINE INFLUENZA VIRUS (EIV)
KENTUCKY 97 AND NON-VACCINATED CONTROLS

| Dog ID | Group^{a} | 0 DPV1^{b} | 0 DPV2 | 13 DPV2 | 8 DPC^{c} |
|---|---|---|---|---|---|
| C5 2801 | 1 | <8 | 64 | 512 | 512 |
| C5 2802 | 1 | <8 | 8 | 128 | 256 |
| C5 2803 | 1 | <8 | 32 | 512 | >1024 |
| C5 2805 | 1 | <8 | 64 | 256 | >1024 |
| C5 2901 | 1 | <8 | 32 | 256 | 512 |
| C5 2903 | 1 | <8 | 64 | 256 | 512 |
| C5 2904 | 1 | <8 | <8 | 64 | 512 |
| C5 3002 | 1 | <8 | 32 | 512 | 512 |
| C5 3003 | 1 | <8 | 16 | 512 | >1024 |
| C5 3004 | 1 | <8 | 64 | 512 | >1024 |
| C5 3006 | 1 | <8 | 64 | 512 | 512 |
| C5 3101 | 1 | <8 | 32 | 512 | >1024 |
| C5 3104 | 1 | <8 | 16 | 512 | >1024 |
| C5 3105 | 1 | <8 | 8 | 256 | 512 |
| C5 3107 | 1 | <8 | 32 | 512 | >1024 |
| C5 3201 | 1 | <8 | 64 | 512 | 512 |
| C5 3202 | 1 | <8 | 8 | 512 | >1024 |
| C5 3203 | 1 | <8 | 16 | 256 | >1024 |
| C5 3204 | 1 | <8 | 8 | 256 | 512 |
| C5 3207 | 1 | <8 | 16 | 512 | 512 |
| C5 3209 | 1 | <8 | 16 | 512 | >1024 |
| C5 2804 | 2 | <8 | <8 | <8 | 128 |
| C5 2806 | 2 | <8 | <8 | <8 | 64 |
| C5 2902 | 2 | <8 | <8 | <8 | 128 |
| C5 3001 | 2 | <8 | <8 | <8 | 128 |
| C5 3005 | 2 | <8 | <8 | <8 | 32 |
| C5 3102 | 2 | <8 | <8 | <8 | 512 |
| C5 3103 | 2 | <8 | <8 | <8 | 64 |
| C5 3106 | 2 | <8 | <8 | <8 | 128 |
| C5 3205 | 2 | <8 | <8 | <8 | 32 |
| C5 3206 | 2 | <8 | <8 | <8 | 64 |
| C5 3208 | 2 | <8 | <8 | <8 | 64 |

^{a}Group 1: dogs vaccinated with a vaccine containing 1500 hemagglutinin (HA) units of EIV Kentucky 97
Group 2: non-vaccinated controls
^{b}DPV: Days post vaccination
^{c}DPC: Days post challenge

### EXAMPLE 3

### Safety of Canine Influenza Virus Vaccine

Animals

A total of one thousand and fifteen (1,015) dogs of any breed and either gender were enrolled in the study. The dogs were six weeks of age or older. Three hundred and nine (309) of the dogs were six to nine weeks of age at the time of the first vaccination. Two hundred and ninety-three dogs (293) were male and nine weeks of age or less. Three hundred and twenty (320) dogs were female and nine weeks of age or less. Six hundred and twenty-two (622) dogs were male and 10 weeks of age or greater. Seven hundred and eighty-five (785) dogs were female and 10 weeks of age or greater. Only animals that were apparently healthy, as determined by a physical examination performed by a veterinarian, were enrolled in the study.

Vaccine

The vaccine was prepared according to standard methods. Each serial of vaccine was stored at 2-7° C. until use.

Vaccination

Only animals that were apparently healthy, as determined by a physical examination by a veterinarian, were vaccinated. The vaccine was administered as a 1 ml dose vaccination by subcutaneous administration followed in three to four weeks by a second 1 ml dose vaccination.

Six hundred and seventy-one (671) dogs were vaccinated with serial 896054A vaccine and three hundred and forty-four (344) dogs were vaccinated with serial 896055A vaccine. One thousand and five (1005) dogs in the study received two doses of vaccine. Ten (10) dogs received only one dose of vaccine and did not complete the full vaccination course.

Statistical Analysis

The estimator is the proportion of local and systemic vaccine reactions.
Prop=nᵣ/Nₜᵥ

where
nᵣ=number of local or systemic reactions
Nₜᵥ=total number of vaccinations administered.

The 95% Clopper-Pearson confidence interval for the proportion of local or systemic reactions was calculated.

This study tested the null hypothesis that the proportion of local or systemic reactions following vaccination are greater than 3%.
H_{O}: pᵥ≧3%
H_{A}: pᵥ<3%

where pv=the proportion of local or systemic reactions following vaccination.

One thousand and fifteen (1015) dogs were enrolled at six veterinary practices in distinct geographic locations. Multiple measurements were taken on each dog over time. Dogs were clustered by veterinary clinic, and hence geographic location.

Because there was only one vaccination group, no baseline assessment is required.

The proportion of local of or systemic reactions as a percent of the total vaccinations administered was calculated. The proportion of local or systemic reactions may be stratified by enrolling site.

All statistical analysis was performed using the SAS system (SAS Institute, Inc.).

Observation of Vaccinated Dogs

Dogs were observed for the incidence of post-vaccination reactions for two weeks following each vaccination. In particular, the veterinarian observed the animal for 30 minutes following vaccination for immediate reactions such as salivation, labored or irregular breathing, shaking, or anaphylaxis. For two weeks after each vaccination, the animals were observed daily for any delayed reactions such as lethargy, anorexia, or unusual swelling at the injection site.

During the observation period following each vaccination, there were no local or systemic adverse reactions reported following two thousand and nineteen (2019) out of two thousand and twenty (2020) doses of vaccine, which represents no reactions in 99.95% (95% Cl 99.72,100) of the vaccinations administered to dogs. A mild systemic reaction (lethargy) was reported in one dog. This dog had lethargy for 24 hours after the first vaccination and then recovered uneventfully without any treatment. The incidence rate for reactions was 0.05% (95% Cl 0.00, 0.28). In total, ten (10) of the original one thousand and fifteen (1,015) dogs enrolled in the study did not complete the full vaccination and observation schedule. In the dogs six to nine weeks of age (the youngest age group) at the time of the first vaccination, the reaction rate was 0.00% (95% Cl 0.00, 0.006) for the 637 doses of vaccine administered by subcutaneous injection under field conditions.

### EXAMPLE 4

The objective of this study is to evaluate the efficacy of the canine influenza vaccine in susceptible puppies by challenging with a virulent canine influenza virus (CIV) strain at three weeks following the administration of the second vaccination.

Animals

Thirty (30) healthy canines will be enrolled in the study. The canines will be either male or female Beagles that are 6 weeks of age. The canines will also be seronegative or have a low antibody titer to CIV.

Animals will be under veterinary care and will be fed a standard commercial diet with water and feed available ad libitum. During the vaccination period, prior to challenge, the puppies will be housed in an isolation facility. Whereas, throughout the challenge observation period, all the puppies will be housed in individual cages in an isolation facility. All housing will be in compliance with applicable animal welfare regulations. Any animal found ill will be reported to the study investigator and/or study director. Concomitant treatment will be administered at the discretion of the supervising veterinarian. No immunosuppressive drugs will be administered within four weeks prior to or post vaccination. Any treatments administered will be documented in the final report.

Vaccine

The vaccine composition will be formulated according to standard methods. The vaccine will be stored at 2° to 7° C. until use.

Challenge

A standard canine influenza virus challenge with a low cell culture passage history will be used as the challenge material.

Experimental Design

The puppies will be randomly sorted into two groups, 20 puppies per vaccinate group and 10 puppies as non-vaccinated controls, using the random number generator in Microsoft® Excel. All puppies will be challenged with the virulent CIV at three weeks post second vaccination.

| Group | Treatment | Route | Dose | No. of Doses | No. of Puppies |
|---|---|---|---|---|---|
| 1 | Vaccinate | S.C. | 1 mL | 2 | 20 |
| 2 | Control | Not | N/A | N/A | 10 |
| | | Applicable | | | |
| | | (N/A) | | | |

Vaccination

The puppies in Group 1 will receive 2 subcutaneous (SC) vaccinations. All SC vaccinations will be administered in the neck region anterior to the shoulder. The time interval between vaccinations will be three weeks. The Group 2, non-vaccinated control puppies, will not receive any vaccine or placebo injection.

Challenge and Observations

Three weeks following the second vaccination, all puppies will be challenged by means of a puppy mask nebulizer (Jorgensen Laboratories) with a total of 10⁷ TCID₅₀ virulent CIV.

Puppies will be observed for nasal exudate, coughing and breathing difficulties daily for three days prior to challenge and 14 days thereafter. Clinical signs will be qualified as mild, moderate or severe and recorded. In addition, rectal temperatures will be monitored and recorded daily for three days prior to the challenge and 14 days thereafter.

Serum Sample Collection

Each puppy will be bled for serum prior to the administration of the first dose of vaccine, on the day of the second vaccination, as well as 7 and 14 days following each vaccination. Following the administration of the second dose of vaccine, the test animals will be bled weekly until challenge.

Each puppy will be bled for serum prior to challenge and at 7, 14 and 21 days following challenge administration.

Sample Collection for Virus Isolation

Pharyngeal swabs will be collected daily from each puppy starting 3 days prior to challenge and for 14 days post challenge and placed in 2 mL of transport medium (MEM supplemented with 0.05% LAH and 2× gentamicin).

Antibody Testing: Canine Influenza Hemagglutination Inhibition Assay

Serum samples may be tested by hemagglutination inhibition assays for (HAI) titers to CIV. The assay will employ 8 HA units of the test indicator virus. All serum samples will be pretreated with periodate and heat inactivated to remove any non-specific inhibitors.

Virus Isolation

Pharyngeal swabs will be thawed and the tubes will be vortexed. Liquid will be expressed from the swabs and the material tested using the MDCK cell line in 96-well plates. Briefly, about 100 μl of sample will be inoculated onto monolayers of MDCK cells in 96-well plates. The cell monolayers will be washed with trypsin containing MEM and 50 μL of sample will be inoculated onto the monolayers of MDCK cells. The inoculum will be allowed to absorb at 35°±20° C. and then an additional 50 μL of trypsin containing MEM added to each well of the plate. At 5 days post inoculation the medium in the plates will be discarded, the monolayers fixed with methanol and stained with a fluorescein labeled specific antibody. The stained monolayers will be evaluated using an ultraviolet microscope and the monolayers scored as positive or negative depending on fluorescence.

Data Analysis

The primary outcome will be prevention of clinical disease. The incidence of clinical signs such as, e.g., sneezing, coughing, nasal discharge, viral shedding, nasal mucoid discharge, etc, will be compared between vaccinates and controls by chi square. If expected cell values are too small, comparisons will be made by Fisher's Exact test. The severity of clinical signs will be compared by Wilcoxon Rank Sum test. Fever will be compared between vaccinates and controls by analysis of variance (ANOVA).

Secondary variables of antibody titer and virus isolation will be assessed. The incidence rate of virus isolation will be compared between vaccinates and controls by chi square. If expected cell values are too small, comparisons will be made by Fisher's Exact test. Antibody titers may be log transformed after assessment of the frequency distributions of the dependent variables. If the residuals are not normally distributed, non-parametric tests will be employed as needed. The level of significance will be set at p<0.05. All statistical analysis will be performed using the SAS system (SAS Institute, Inc.).

Data Interpretation

For the study to be valid, all puppies designated as controls must remain either sero-negative or have no significant increase in antibody titer for the test antigens.

The incidence and severity of clinical disease as assessed by clinical signs, fever and virus isolation must be significantly lower in vaccinates compared to controls.

While the invention has been described in each of its various embodiments, it is expected that certain modifications thereto may be undertaken and effected by the person skilled in the art without departing from the true spirit and scope of the invention, as set forth in the previous description and as further embodied in the following claims.

What is claimed is:

1. A composition effective at treating, preventing or ameliorating canine influenza virus (CIV) infection in a canine, comprising one or more canine influenza virus (CIV) or equine influenza virus (EIV) immunogen.

2. The composition of claim 1, further comprising a pharmacologically acceptable carrier, excipient, diluent, or preservative.

3. The composition of claim 1, further comprising an adjuvant.

4. The composition of claim 1, further comprising at least one additional vaccine immunogen.

5. The composition of claim 4, wherein the at least one additional vaccine immunogen is a vaccine immunogen against a canine affliction.

6. The composition of claim 1, wherein said CIV or EIV immunogen is isolated from a canine with clinical symptoms of flu disease from infection with a pathogenic EIV or CIV strain.

7. The composition of claim 6, wherein said EIV strain is Ohio 03.

8. The composition of claim 6, wherein said EIV or CIV strain causes virus shedding in greater than 60% of animals challenged with said strain.

9. The composition of claim 1, wherein the immunogen is live, attenuated, or killed.

10. The composition of claim 9, wherein the immunogen is attenuated and has been serially passaged.

11. A method of generating an equine influenza virus (EIV) or canine influenza virus (CIV) immunogen comprising infecting a canine with a strain of equine influenza virus or canine influenza virus sufficient to cause flu disease in said canine, and then isolating an EIV or CIV immunogen from said canine and cultivating said immunogen.

12. A method of protecting a canine from canine influenza virus disease comprising administering the compositions of claim 1 to the canine.

13. The method of claim 12, wherein the composition is administered in one dose.

14. The method of claim 12, wherein the composition is administered in two or more doses.

15. A challenge model useful in assessing the efficacy of a composition against canine influenza virus, comprising an equine influenza virus (EIV) or canine influenza virus (CIV) strain that has been isolated from a canine.

16. A vaccine composition effective at treating, preventing or ameliorating canine influenza virus (CIV) infection in a canine, comprising one or more canine influenza virus (CIV) or equine influenza virus (EIV) immunogen.

17. The vaccine composition of claim 16, further comprising a pharmacologically acceptable carrier, excipient, diluent, or preservative.

18. The vaccine composition of claim 16, further comprising an adjuvant.

19. The vaccine composition of claim 16, further comprising at least one additional vaccine immunogen.

20. The vaccine composition of claim 19, wherein the at least one additional vaccine immunogen is a vaccine immunogen against a canine affliction.

21. The vaccine composition of claim 16, wherein said CIV or EIV immunogen is isolated from a canine with clinical symptoms of flu disease from infection with a pathogenic EIV or CIV strain.

22. The vaccine composition of claim 21, wherein said EIV strain is Ohio 03.

23. The vaccine composition of claim 21, wherein said EIV or CIV strain causes virus shedding in greater than 60% of animals challenged with said strain.

24. The vaccine composition of claim 16, wherein the immunogen is live, attenuated, or killed.

25. The vaccine composition of claim 24, wherein the immunogen is attenuated and has been serially passaged.
